(19) United States
(12) Plant Patent Application Publication
(10) Pub. No.: US 2004/0205873 P1
(43) Pub. Date: Oct. 14, 2004

(54) Osteospermum plant named of 'Sunny Amanda'

(76) Inventor: Bjarne N. Larsen, Odense N (DK)

Correspondence Address: FOLEY AND LARDNER, SUITE 500, 3000 K STREET NW, WASHINGTON, DC, 20007, US

(21) Appl. No.: 10/788,297

(22) Filed: Mar. 1, 2004

(30) Foreign Application Priority Data
Jul. 3, 2002 (EP) 2002/0999

(51) Int. Cl. A01H 5/00

(52) U.S. Cl. PLT/360

(57) ABSTRACT

A new distinct cultivar of Osteospermum plant named ‘Sunny Amanda’, characterized by its white flower color RHS 157A, with yellow tinge at the apex RHS 163B, and almost golden undersides; gray-purple disks RHS N187D; dense and bushy plant form; with globular plant habit; moderately vigorous growth habit, but less need for chemical growth retardation, and ovate to lanceolate leaves.

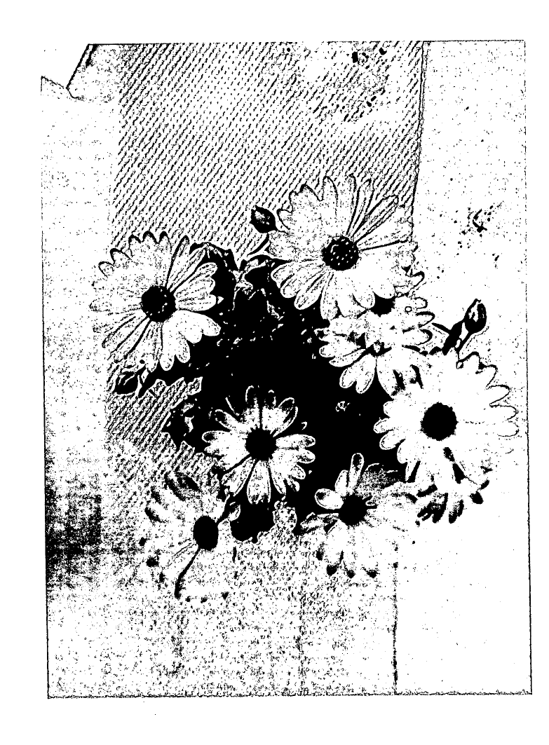

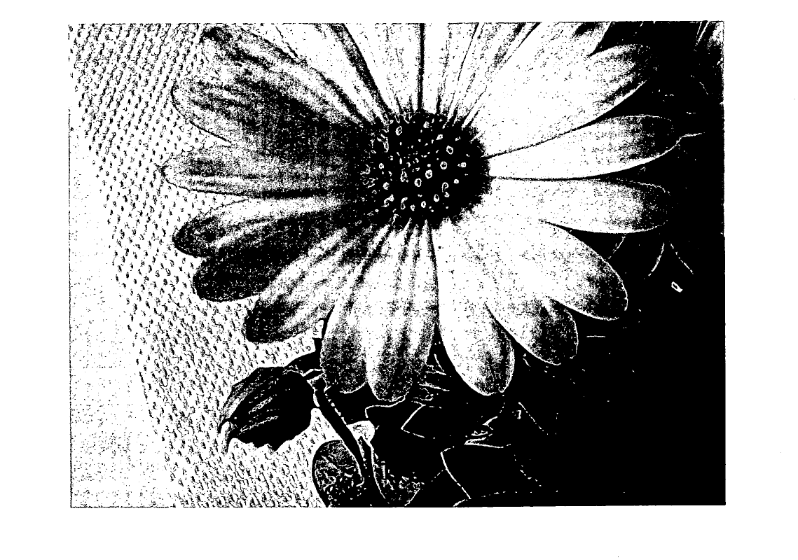

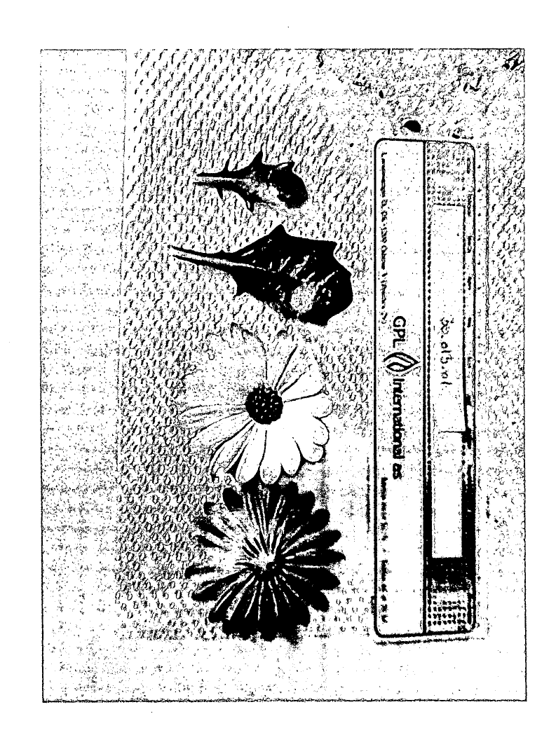

### GENUS AND SPECIES OF THE PLANT CLAIMED

Osteospermum eclonis (DC) T. Norl.

### VARIETY DENOMINATION

Sunny Amanda

### BACKGROUND OF THE INVENTION

The present Invention relates to a new and distinct cultivar of Osteospermum plant, botanically known as Osteospermum eclonis (DC) T. Norl., commonly known by the name Cape Daisy, and hereinafter referred to by the name ‘Sunny Amanda’.

The new Osteospermum is a product of a planned breeding program conducted by the Inventor, Bjarne Larsen, in Stige, Denmark. The Inventor selected the new Osteospermum cultivar in 2002 on the basis of its flower color and compact, freely branching habit. Plants of the new Osteospermum are more upright, compact and have a unique color combination of upper and underside of the ray petals.

Asexual reproduction of the new cultivar by terminal vegetative cuttings taken and propagated during trial production batches in Stige, Denmark, has shown that the unique features of this new Osteospermum are stable and reproduced true to type in many successive generations.

### BRIEF SUMMARY OF THE INVENTION

Plants of the cultivar ‘Sunny Amanda’ have not been observed under all possible environmental conditions. The phenotype may vary somewhat with variations in environment such as temperature, light intensity, day length, and fertility level without, however, any variance in genotype.

The following traits have been repeatedly observed and are determined to be the unique characteristics of ‘Sunny Amanda’. These characteristics in combination distinguish ‘Sunny Amanda’ as a new and distinct cultivar:

1. White flower colors RHS 157A with yellow tinge at the apex RHS 163B, and almost golden undersides; gray-purple disks RHS N187D.

2. Dense and bushy plant form; with globular plant habit.

3. Moderately vigorous growth habit, but less need for chemical growth retardation.

4. Ovate to lanceolate leaves.

Plants of the cultivar ‘Sunny Amanda’ can be compared to plants of the Osteospermum eclonis (DC) T. Norl. cultivar ‘Sunny Alex’ (unpatented). In side-by-side comparisons conducted by the Inventor in Stige Denmark, plants of the cultivar ‘Sunny Amanda’ and the cultivar ‘Sunny Alex’ differ in the following characteristics:

1. ‘Sunny Amanda’ has striking color combinations of white ray flowers with yellow tinge at the apex RHS 163B, and gray-purple disk with orange pollen, while ‘Sunny Alex’ is orange yellow.

2. ‘Sunny Amanda’ has entire, ovate to lanceolate leaves while the leaves of ‘Sunny Alex’ are broadly lobed.

3. ‘Sunny Amanda’ has longer, but sturdier peduncles than plants of the cultivar ‘Sunny Alex’.

4. ‘Sunny Amanda’ is globular and more compact than the upright plants of the cultivar ‘Sunny Alex’.

5. ‘Sunny Amanda’ has fewer and larger flowers per plant than the plants of the cultivar ‘Sunny Alex’.

### BRIEF DESCRIPTION OF THE PHOTOGRAPHS

The accompanying color photographs illustrate the overall appearance and details of flower form, color and structures of the new cultivar, showing the colors as true as it is reasonably possible to obtain in color reproductions of this type. Colors in the photographs may differ slightly from the color values cited in the detailed botanical description, which more accurately describe the actual colors of the new Osteospermum.

The first photograph is a top view of a typical flowering plant of ‘Sunny Amanda’ as grown in an 11 cm pot. The second photograph is a close-up of the composite flower of ‘Sunny Amanda’. The third photograph shows the flowers and leaves of ‘Sunny Amanda’ (in the third photograph, ‘Sunny Amanda’ is labelled by its breeder's reference no. ‘30.013.01’).

### DETAILED BOTANICAL DESCRIPTION

In the following description, color references are made to The Royal Horticultural Society Colour Chart, 4^{th} edition, where general terms of ordinary dictionary significance are used. Plants were grown under greenhouse conditions. Plants used for this description were grown for about 20 weeks after cutting.

Plant description:

Form.—Perennial plant with globular plant habit. Composite flower heads. Freely branching with lateral flowering branches forming at every node; dense and bushy.

Crop time.—After rooting, about 20 weeks are required to produce finished flowering plants in 11 cm pots.

Plant height (soil level to top of plant plane).—About 20 cm.

Vigor.—Moderately vigorous growth rate.

Time to initiate roots.—About 10 to 14 days at 18 to 21 C in tunnels in a greenhouse.

Root description.—Fine, well branched.

Foliage description.—Leaves alternate, single, slightly lobed, brochidodromus venation.

Length.—3-5 cm. Width: About 30 mm. Shape: Obovate. Apex: obtuse. Base: attenuate.

Margin.—broadly dentate. Texture: smooth, glabrous, shiny. Scattered short, stiff hairs. Color: Young foliage, upper surfaces 147A, yellow-green and lower surface 148C. Mature foliage, upper surface 139B and lower surface 147A. Venation, 148B.

Flower description:

Flower arrangement and shape.—Tubular disk and petalled ray flowers in composite daisy capitulae; with 15 parted involucre. 10 cm sturdy peduncles.

Natural flowering season.—Continuous throughout the spring and summer in temperate regions. Season can be extended by vernalization and long day treatments.

Flower longevity on the plant.—5 to 9 days; longevity of individual flowers is highly dependent on temperature and light conditions. Flowers persistent, petals folding and withering slowly.

Inflorescence diameter.—About 7 cm.

Flowers.—Length: Ray petals: About 27 mm. Width: About 8 mm. Lanceolate to elliptic, acute ray petal lobes. Ray petal color: Upper surfaces 157D to 163B at the apex, white to gray-orange; underside 162D to 161A with stripes of 177A, gray-orange, under some light conditions the underside appears golden. Disk flowers N187D gray purple with white undersides.

Peduncle.—Strength: strong. Length: About 10 cm. Diameter: About 2 mm. Color: 144C yellow-green.

Reproductive organs:

Anthers.—Linear fused, stamen color 202A, black.

Pollen.—N25A orange.

Pistil and stigma.—Color N187B, purple.

Seed.—Length: About 1 mm. Diameter: About 0.3 mm.

Weather tolerance.—Plants of the new Osteospermum have exhibited good tolerance to draught, rain and wind, however flowering may cease during hot periods above 30 C.

I claim:

1. A new and distinct cultivar of Osteospermum plant named ‘Sunny Amanda’, as illustrated and described herein.
